(19) United States
(12) Patent Application Publication
(10) Pub. No.: US 2023/0303770 A1
(43) Pub. Date: Sep. 28, 2023

(54) POLYOL COMPOUNDS AND ADHESIVE COMPOSITIONS PREPARED WITH THE SAME

(71) Applicants: Dow Global Technologies LLC, Midland, MI (US); Rohm and Haas Company, Collegeville, PA (US)

(72) Inventors: Ke Shi, Shanghai (CN); Chenyan Bai, Shanghai (CN); Zhaohui Qu, Shanghai (CN); Tao Wang, Shanghai (CN); Cheng Shen, Shanghai (CN)

(21) Appl. No.: 18/004,805

(22) PCT Filed: Jul. 14, 2020
(86) PCT No.: PCT/CN2020/101838

§ 371 Date: Jan. 9, 2023

(51) Int. Cl. C08G 65/331; C07C 271/64; C07C 269/06; C08G 18/10; C08G 18/50; C09J 175/08; C09J 5/06; B32B 37/12; B32B 7/12; B32B 27/36; B32B 27/32; B32B 27/08

(52) U.S. Cl. CPC C08G 65/331; B32B 7/12; B32B 27/08; B32B 27/32; B32B 27/36; B32B 37/12; C07C 269/06; C07C 271/64; C08G 18/10; C08G 18/5045; C09J 5/06; C09J 175/08; B32B 2037/1253; B32B 2250/02; B32B 2255/26; B32B 2307/31; B32B 2307/748; C09J 2475/00

(57) ABSTRACT

A unique polyol compound formed by a reaction between a carbonate compound represented by Formula (I) and a polyamine compound is provided. A polyurethane-based adhesive composition comprising the polyol compound can produce an adhesive layer exhibiting superior bond strength and heat seal strength which will not be substantially deteriorated by the change in the weight ratio between isocyanate component and polyol component. Methods for preparing the polyol compound and for preparing the adhesive composition, as well as a laminate article prepared with said adhesive composition are also provided.

### FIELD OF THE INVENTION

The present disclosure relates to a novel polyol compound and a method for preparing the same, an adhesive composition comprising said polyol compound and a method for preparing the same, a laminate product comprising an adhesive layer derived from the adhesive composition and a method for preparing the same. The adhesive layer prepared with said adhesive composition exhibits high tolerance to the change in composition and can achieve good bond strength and heat seal strength which will not be substantially deteriorated by the change in the weight ratio between the isocyanate component and the polyol component.

### BACKGROUND

Adhesive compositions are useful for a wide variety of applications. For instance, they can be used to bond substrates such as polyethylenes, polypropylenes, polyesters, polyamides, metals, papers, or cellophanes to form composite films, i.e., laminates. The use of adhesives in different laminating end-use applications is generally known. For example, adhesives can be used in the manufacture of film/film and film/foil laminates commercially used in the packaging industry.

Among many such known systems, the use of polyurethane based laminating adhesives is preferred because of their many desirable properties including good adhesion, peel strength, heat seal strength and resistance to aggressive filling goods. However, it is desirable to develop a new adhesive system whose polyol is prepared with precise synthetic route and appointed composition, and thus can exhibit desirable properties with no substantial flunctation.

After persistent exploration, we have surprisingly developed a polyurethane adhesive composition which can achieve one or more of the above targets.

### SUMMARY OF THE INVENTION

The present disclosure provides a unique polyol compound and a polyurethane adhesive composition comprising the same.

In a first aspect of the present disclosure, the present disclosure provides a polyol compound formed by a reaction between a carbonate compound represented by Formula I and a polyamine compound comprising at least two amine groups.

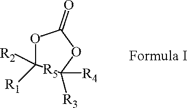

wherein each of R₁ R₂, R₃ and R₄ are identical with each other or different from each other, and are independently selected from the group consisting of hydrogen, linear or branched C₁ to C₆ alkyl, linear or branched C₁₋C_{6,} alkoxy, hydroxyl, halogen and hydroxyl substituted-C₁-C₆ alky, or R₁ is directly linked with R₃ so that a combination of R₁, R₃, R₅ and the carbon atoms attached thereto form a C₅-C₈ cycloalkyl group substituted with at least one hydroxyl, according to a preferable embodiment of the present disclosure, R₁ is directly linked with R₃ so that the combination of R₁, R₃, R₅ and the carbon atoms attached thereto form a cyclic radical selected from the group consisting of mono-hydroxyl substituted cyclopentyl, di-hydroxyl hydroxyl substituted cyclopentyl, tri-hydroxyl substituted cyclopentyl, mono-hydroxyl substituted cyclohexyl, di-hydroxyl substituted cyclohexyl, tri-hydroxyl substituted cyclohexyl, tetra-hydroxyl substituted cyclohexyl, mono-hydroxyl substituted cycloheptyl, di-hydroxyl substituted cycloheptyl, tri-hydroxyl substituted cycloheptyl, tetra-hydroxyl substituted cycloheptyl, mono-hydroxyl substituted cyclooctyl, di-hydroxyl substituted cyclooctyl, tri-hydroxyl substituted cyclooctyl, and tetra-hydroxyl substituted cyclooctyl,
R₅ is a direct covalent bond, a methylene group, a 1,2-ethylene group, or a 1,3-propylene group, wherein the methylene group, 1,2-ethylene group and 1,3-propylene group are optionally substituted with at least one C₁-C₆ alkyl, C₁-C₅ alkoxy, hydroxyl, halogen and hydroxyl-C₁-C₆ alky. According to a preferable embodiment of the present disclosure, the carbonate compound of Formula 1 has a hydroxyl functionality of at least 1.0, 1.2, or at least 1.5, or at least 1.6, or at least 1.8, or at least 2.0, or at least 2.2, or at least 2.5, or at least 2.8, or at least 3.0. According to another embodiment of the present disclosure, the carbonate compound comprises at least one primary hydroxyl group, preferably comprises at least two primary hydroxyl groups, more preferably at least three primary hydroxyl groups. According to another embodiment of the present disclosure, said polyol compound of the present disclosure comprises at least two hydroxyl groups, or comprises at least three groups, or comprises at least four hydroxyl groups.

According to another preferable embodiment of the present disclosure, the carbonate compound is selected from the group consisting of glycerin carbonate, 1-methyl-glycerin carbonate, 2-methyl-glycerin carbonate, 3-methyl-glycerin carbonate, 1,1-dimethyl-glycerin carbonate, 2.2-dimethyl-glycerin carbonate, 1,2-dimethyl-glycerin carbonate, 1-ethyl-glycerin carbonate, 1,1-diethyl-glycerin carbonate, 2-ethyl-glycerin carbonate, 2,2-diethyl-glycerin carbonate, I-propyl-glycerin carbonate, 1-butyl-glycerin carbonate, 1,2,3,4-tetrahydroxybutane carbonate, 1,2,3,4,5-pentahydroxypentane carbonate, 1,2,3,4,5,6-hexahydroxyhexane carbonate, trimethyolmethane carbonate, trimethyolethane carbonate, trimethylolpropane carbonate, pentaerythritol carbonate, and combinations thereof. According to another preferable embodiment of the present disclosure, the polyamine compound is selected from the group consisting of piperazine, C₂-C₁₆ aliphatic diamine or triamine, C₄-C₁₅ cycloaliphatic or aromatic diamine or triamine, C₇-C₁₅ araliphatic diamine or triamine, diaminated or triaminated C₂-C₈ aliphatic alcohol, polyether diamine, polyester diamine, and combinations thereof. According to another preferable embodiment of the present disclosure, the reaction between the carbonate compound and the polyamine compound occurs with a molar ratio between the carbonate compound and the amine group in the polyamine compound of 5:1 to 1:5; such as from 4:1 to 1:4; or from 3:1 to 1:3; or from 2:1 to 1:2; or from 1.5:1 to 1: 1.5; or at a ratio of about 1:1.

In a second aspect of the present disclosure, the present disclosure provides a method for preparing the polyol compound of the present disclosure, comprising a step of reacting a carbonate compound represented by Formula 1 with a polyamine compound comprising at least two amine groups,

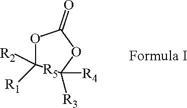

wherein each of R₁, R₂, R₃ and R₄ are identical with each other or different from each other, and are independently selected from the group consisting of hydrogen, linear or branched C₁ to C₆ alkyl, linear or branched C₁-C₅ alkoxy, hydroxyl, halogen and hydroxyl-C₁₋C₆ alky, or R₁ is directly linked with R₃ so that a combination of R₁, R₃, R₅ and the carbon atoms attached thereto forms a C₅-C₈ cycloalkyl group substituted with at least one hydroxyl group, and, and
R₅ is a direct covalent bond, a methylene group, a 1,2-ethylene group, or a 1,3-propylene group, wherein the methylene group. 1,2-ethylene group and 1,3-propylene group are optionally substituted with C₁-C₆ alkyl, C₁-C₅ alkoxy, hydroxyl, halogen and hydroxyl-C₁₋ C₆ alky.

In a third aspect of the present disclosure, the present disclosure provides an adhesive composition, comprising: (A) an isocyanate component comprising a prepolymer having two or more free isocyanate groups; and (B) an isocyanate-reactive component comprising the polyol compound of the present disclosure.

Preferably, the adhesive composition comprises any one or any combinations of the following features: the adhesive composition is solventless or may comprise solvent; the weight ratio between the prepolymer and the polyol compound is from 100:20 to 100:100; the isocyanate-reactive component further comprises at least one second polyol compound selected from the group consisting of polycarbonate polyol, polyether polyol, polyester polyol, and combinations thereof; the second polyol compound has a hydroxyl functionality of at least 1.2, or at least 1.5, or at least 1.6, or at least 1.8, or at least 2.0, or at least 2.2, or at least 2.5, or at least 2.8, or at least 3.0; the content of the polyol compound of the present disclosure is from 40 wt% to 80 wt%, and the content of the second polyol compound is from 20 wt% to 60 wt%, based on the total weight of the (B) polyol component; the (A) isocyanate component has an average isocyanate functionality larger than 1.1, such as at least 1.5, or at least 1.8, can be up to 6.0, or up to 5.5, or up to 5.0. or up to 4.5, or up to 4.0, or up to 3.5, or up to 3.0, or up to 2.5, or up to 2.0, or up to 1.8, or up to 1.5, the prepolymer comprises at least two unreacted (free) isocyanate groups and is derived from the reaction of an isocyanate compound (preferably a monomeric isocyanate compound) with at least one second isocyanate-reactive compound, wherein the (monomeric) isocyanate compound is selected from the group consisting of C₄-C₁₂ aliphatic isocyanate comprising at least two isocyanate groups, C₆-C₁₅ cycloaliphatic or aromatic isocyanate comprising at least two isocyanate groups, C₇-C₁₅ araliphatic isocyanate comprising at least two isocyanate groups, carbodiimide modified isocyanates, and a combination thereof, and the second isocyanate-reactive compound can be selected from the group consisting of C₂-C₁₆ aliphatic polyhydric alcohol comprising at least two hydroxyl groups. C₆-C₁₅ cycloaliphatic or aromatic polyhydric alcohol comprising at least two hydroxyl groups, C₇-C₁₅ araliphatic polyhydric alcohol comprising at least two hydroxyl groups, polyester polyol, polyether polyol, polycarbonate polyol and combinations thereof. According to an embodiment of the present disclosure, the prepolymer can also be prepared by using the polyol compound of the present disclosure. According to a preferable embodiment of the present disclosure, the prepolymer of the (A) isocyanate component has an average isocyanate functionality of larger than 1.1, such as at least 1.5, or at least 1.8, can be up to 6.0, or up to 5.0, or up to 4.0, or up to 3.0, or up to 2.0.

In a fourth aspect of the present disclosure, the present disclosure provides a method for preparing the adhesive composition of the present disclosure, comprising the steps of
(a) providing the isocyanate component, and
(b) providing the isocyanate-reactive component, wherein the isocyanate-reactive component comprises a polyol compound formed by reacting the carbonate of Formula I with the polyamine.

According to various embodiments of the present disclosure, the adhesive composition is a two-component adhesive, wherein the isocyanate component and the isocyanate-reactive component are stored and transported in separate packages, and are combined immediately before being applied to any objects.

In a fifth aspect of the present disclosure, the present disclosure provides a method for preparing a laminate article with the adhesive composition of the present disclosure, comprising the steps of providing a first substrate and a second substrate, mixing the isocyanate component with the isocyanate-reactive component to form a curable mixture; adhering the first substrate to the second substrate by using a layer of the curable mixture; and curing the curable mixture, or allowing it to cure.

In a sixth aspect of the present disclosure, the present disclosure provides a laminate article comprising at least two substrates and an adhesive layer sandwiched therebetween, wherein the adhesive layer is formed by the reaction between the (A) isocyanate component and the (B) isocyanate-reactive component of the adhesive composition.

It is to be understood that both the foregoing general description and the following detailed description are exemplary and explanatory only and are not restrictive of the invention, as claimed.

### BRIEF DESCRIPTION OF THE DRAWINGS

FIG. 1 shows the mechanism of the reaction between glycerin carbonate and polyamine;

FIG. 2 shows the GPC characterization results of polyol compounds according to two embodiments of the present disclosure.

### DETAILED DESCRIPTION OF THE INVENTION

Unless defined otherwise, all technical and scientific terms used herein have the same meaning as commonly understood by one of ordinary skill in the art to which the invention belongs. Also, all publications, patent applications, patents, and other references mentioned herein are incorporated by reference.

As disclosed herein, “and/or” means “and, or as an alternative”. All ranges include endpoints unless otherwise indicated.

As disclosed herein, unless indicated otherwise, the term “polyol compound” or “polyol compound according to the present disclosure” specifically refer to the novel polyol compound developed by the present disclosure. In the context of the present disclosure, all the polyol compounds other than the above stated novel polyol compound developed by the present disclosure will be referred as “the second polyol compounds”, “other polyol compounds” or “additional polyol compounds”.

According to various embodiments of the present disclosure, the adhesive composition is a “two-part” or “two-package” composition comprising an isocyanate component (A) and an isocyanate-reactive component (B) comprising the polyol compound of the present disclosure, which is derived from the reaction between a carbonate represented by Formula 1 and a polyamine. The particularly defined polyol compound of the present disclosure can properly impart desirable properties to the adhesive composition and the adhesive layer prepared therefrom. According to a preferable embodiment, the isocyanate component (A) and the isocyanate-reactive component (B) are transported and stored separately, combined shortly or immediately before being applied during the manufacture of the laminate article.

### The Isocyanate Component (A)

According to an embodiment of the present disclosure, the isocyanate component (A) has an average NCO functionality of at least about 1.5, preferably from about 2 to about 10, more preferably from about 2 to about 8, more preferably from about 2 to about 6, and most preferably about 2. Preferably, the isocyanate component (A) has an average NCO functionality of 2.0.

In some embodiments, the isocyanate component includes a prepolymer formed by the reaction of (i) one or more (monomeric or oligomeric) isocyanate compounds comprising at least two isocyanate groups with (ii) one or more second isocyanate-reactive compounds having at least two isocyanate-reactive groups; wherein the prepolymer comprises at least two free isocyanate groups. According to a preferable embodiment, the second isocyanate compound used for preparing the above stated prepolymer is selected from the group consisting of C₄-C₁₂ aliphatic isocyanates comprising at least two isocyanate groups, C₆-C₁₅ cycloaliphatic or aromatic isocyanates comprising at least two isocyanate groups, C₇-C₁₅ araliphatic isocyanates comprising at least two isocyanate groups, and combinations thereof; and is more preferably selected from the group consisting of m-phenylene diisocyanate, 2,4-toluene diisocyanate and/or 2,6-toluene diisocyanate (TDI), the various isomers of diphenylmethanediisocyanate (MDI), carbodiimide modified MDI products, hexamethylene-1,6-diisocyanate, tetramethylene-1,4-diisocyanate, cyclohexane-1,4-diisocyanate, hexahydrotoluene diisocyanate, hydrogenated MDI, naphthylene-1,5-diisocyanate, isophorone diisocyanate (IPDI), isomers of naphthalene-dipolyisocyanate (“NDI”) such as 1, 5-NDI, isomers of hexamethylene dipolyisocyanate (“HDI”), isomers of isophorone dipolyisocyanate (“IPDI”), isomers of xylene dipolyisocyanate (“XDI”), or mixtures thereof. According to another preferable embodiment of the present disclosure, the second isocyanate-reactive compound used for preparing the above stated prepolymer is selected from the group consisting of monomeric polyfunctional alcohols, such as C₂-C₁₆ aliphatic polyhydric alcohols comprising at least two hydroxyl groups, C₆-C₁₅ cycloaliphatic or aromatic polyhydric alcohols comprising at least two hydroxyl groups, C₇-C₁₅ araliphatic polyhydric alcohols comprising at least two hydroxyl groups; and polymeric polyols, such as polyester polyols, polyether polyols, polycarbonate polyols, a blend of said polyester polyols and polyether polyols, and a combination thereof. According to another embodiment of the present disclosure, the second isocyanate-reactive compounds used for preparing the above said prepolymer can be the polyol compound of the present disclosure.

Compounds having isocyanate groups, such as the above stated prepolymer and the monomeric or oligomeric isocyanate compound used for preparing the prepolymer, may be characterized by the parameter “%NCO” which is the amount of isocyanate groups by weight based on the weight of the compound. The parameter %NCO can be measured by the method of ASTM D 2572-97 (2010). According to an embodiment of the present disclose, the disclosed prepolymer and the monomeric or oligomeric isocyanate compound used for preparing the prepolymer have a %NCO of at least 3 wt%, or at least 5 wt%, or at least 7 wt%, or have a % NCO not exceeding 40 wt%, 35 wt%, 30 wt %, or 25 wt%, or 22 wt%, or 20 wt%.

According to an embodiment of the present disclosure, the content of the (monomeric or oligomeric) isocyanate compound used for preparing the prepolymer can be from 30 wt% to 65 wt%, with the total weight of the isocyanate component (A) being taken as 100 wt%. According to a preferable embodiment of the present disclosure, the content of the (monomeric or oligomeric) isocyanate compound used for preparing the prepolymer can be in the numerical range obtained by combining any two of the following end point values: 27 wt%, 30 wt%, 33 wt%, 35 wt%, 40 wt%, 45 wt%, 50 wt%, 55 wt%, 60 wt%, 65 wt% and 70 wt%.

According to another preferable embodiment of the present disclosure, the content of the second isocyanate-reactive compound used for preparing the prepolymer can be in the numerical range obtained by combining any two of the following end point values: 8 wt%, 10 wt%, 12 wt%, 15 wt%, 18 wt%, 20 wt%, 22 wt%, 25 wt%, 28 wt%, 30 wt%, 32 wt%, 35 wt%, 37 wt%, 40 wt%, 42 wt%, 45 wt%, 48 wt%, 50 wt%, 52 wt%, 54 wt%, 55 wt%, 57 wt%, 60 wt%, 62 wt%, 65 wt%, 67 wt%, 70 wt%, 72 wt%, 75 wt%, 80 wt%, 82 wt% and 85 wt%, with the total weight of the isocyanate component (A) being taken as 100 wt%.

According to another preferable embodiment of the present disclosure, the polyol compound of the present disclosure, i.e. the polyol compound formed by the reaction between the carbonate of Formula I and a polyamine is used as part or all of the isocyanate-reactive compound used for preparing the above said prepolymer. According to a preferable embodiment of the present disclosure, the content of the polyol compound of the present application in the isocyanate-reactive compound for preparing the above said prepolymer is from 0 wt% to 100 wt%, or from 10 wt% to 100 wt%, or from 20 wt% to 100 wt%, or from 25 wt% to 95 wt%, or from 30 wt% to 80 wt%, or can be in the numerical range obtained by combining any two of the following end point values: 0 wt%, 2 wt%, 5 wt%, 8 wt%, 10 wt%, 12 wt%, 15 wt%, 20 wt%, 22 wt%, 25 wt%, 28 wt%. 30 wt%, 35 wt%, 40 wt%, 42 wt%, 45 wt%, 48 wt%. 50 wt%, 52 wt%, 55 wt%, 58 wt%, 60 wt%, 62 wt%, 63 wt%, 65 wt%, 68 wt%, 70 wt%, 72 wt%, 74 wt%, 75 wt%, 77 wt%, 78 wt%, 80 wt%, 82 wt%. 85 wt%, 88 wt%, 90 wt%, 92 wt%. 95 wt%, 98 wt%, 99 wt% and 100 wt%, with the total weight of all the isocyanate-reactive compounds used for preparing the prepolymer being taken as 100 wt%.

### The Isocyanate-Reactive Component (B)

According to various embodiments of the present disclosure, the isocyanate-reactive component comprises a polyol compound derived from the reaction between a carbonate compound of Formula I and a polyamine, and optionally one or more additional polyol compounds other than said polyol compound of the present application, which will be referred as the second polyol compound hereinafter.

According to a preferable embodiment of the present disclosure, the isocyanate-reactive component (B) does not comprise the second polyol compound and exclusively comprises the polyol compound formed by the reaction between the carbonate of Formula 1 and a polyamine.

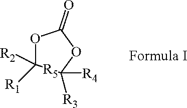

wherein each of R₁, R₂, R₃ and R₄ are identical with each other or different from each other, and are independently selected from the group consisting of hydrogen, linear or branched C₁ to C₆ alkyl, linear or branched C₁-C₅ alkoxy, hydroxyl, halogen and hydroxyl-C₁₋C₆ alky, or R₁ is directly linked with R₃ so that a combination of R₁, R₃, R₅ and the carbon atoms attached thereto forms a C₅-C₈ cycloalkyl group substituted with at least one hydroxyl, and
R₅ is a direct covalent bond, a methylene group, a 1,2-ethylene group, or a 1,3-propylene group, wherein the methylene group, 1,2-ethylene group and 1.3-propylene group are optionally substituted with C₁-C₆ alkyl, C₁-C₅ alkoxy, hydroxyl, halogen and hydroxyl-C₁₋C₆ alky.

According to an embodiment of the present application, the carbonate of Formula 1 can be selected from the group consisting of glycerin carbonate, 1-methyl-glycerin carbonate, 2-methyl-glycerin carbonate, 3-methyl-glycerin carbonate, 1,1-dimethyl-glycerin carbonate, 2,2-dimethyl-glycerin carbonate, 1,2-dimethyl-glycerin carbonate, 1-ethyl-glycerin carbonate, 1,1-diethyl-glycerin carbonate, 2-ethyl-glycerin carbonate, 2,2-diethyl-glycerin carbonate, 1-propyl-glycerin carbonate, 1-butyl-glycerin carbonate, 1,2,3,4-tetrahydroxybutane carbonate, 1,2,3,4,5-pentahydroxypentane carbonate, 1,2,3,4,5,6-hexahydroxyhexane carbonate, trimethyolmethane carbonate, trimethyolethane carbonate, trimethylolpropane carbonate, pentaerythritol carbonate, and combinations thereof.

According to another preferable embodiment of the present disclosure, the polyamine compound is selected from the group consisting of piperazine, C₂-C₁₆ aliphatic diamine (such as ethylene diamine, propylene diamine, butylene diamine, pentamethylene diamine, hexamethylene diamine) or triamine (such as 1,2,3-triamine propane, 1,2,3-triamine butane, 1,2,4-triarnine butane, etc.), C₄-C₁₅ cycloaliphatic or aromatic diamine or triamine (such as 1,2-diaminocyclohexane, 1,3-diaminocyclohexane, 1,4-diaminocyclohexane, 1,2,3-triamino-cyclohexane, 1,2,4-triamino-cyclohexane, 1,2-diaminobenzene, 1,3-diaminobenzene, 1,4-diaminobenzene, 1,2,3-triamino-benzene, 1,2,4-triamino-benzene, etc.), C₇-C₁₅ araliphatic diamine or triamine (such as p-xylenediamine, or o-xylenediamine), diaminated or triaminated C₂-C₈ aliphatic alcohol (such as aminoethylethanolamine), polyether diamine, polyester diamine, and combinations thereof. According to another preferable embodiment of the present disclosure, the reaction between the carbonate compound of Formula 1 and the polyamine compound occurs with a molar ratio between the carbonate compound and the amine group in the polyamine compound of 5:1 to 1:5; such as from 4:1 to 1:4; or from 3:1 to 1:3; or from 2:1 to 1:2; or from 1.5:1 to 1:1.5: or at a ratio of about 1:1.

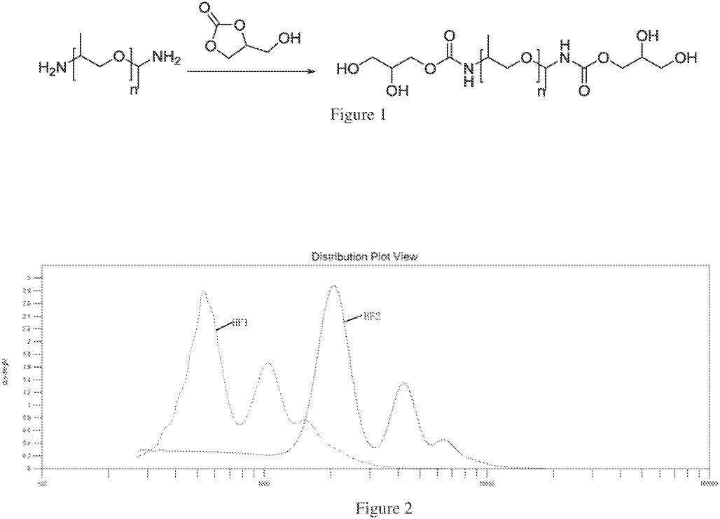

A reaction mechanism for preparing the polyol compound of the present disclosure is illustratively shown in FIG. 1, wherein glycerin carbonate and a polyether diamine are selected as exemplary reactants, but the protection scope of the present application is not limited thereby.

In the reaction shown in FIG. 1, the polyether amine has a polymerization degree n of about 5 to 50, such as from 6 to 45, or from 7 to 42, or from 8 to 35. According to a preferable embodiment of the present disclosure, the polyether amine has a number average molecular weight (Mn) of about 100 to 5,000 g/mol, such as from 200 to 4.000 g/mol, or from 300 to 3,000 g/mol, or from 400 to 2,000 g/mol; or can be in the numerical range obtained by combining any two of the following end point values: 500 g/mol, 600 g/mol, 700 g/mol, 800 g/mol, 900 g/mol, 1,000 g/mol, 1,100 g/mol, 1,200 g/mol, 1,300 g/mol, 1,400 g/mol, 1,500 g/mol, 1,600 g/mol, 1,700 g/mol, 1,800 g/mol, 1,900 g/mol. According to a preferable embodiment of the present disclosure, the polyether amine has an amine functionality of about 1.5 to 8.0. such as from 1.8 to 6.0, or from 1.9 to 5.0, or from 2.0 to 4.0, or from 2.0 to 3.5, or from 2.0 to 3.0, or from 2.0 to 2.5 or from 2.0 to 2.2, or from 2.0 to 2.1, and is most preferably 2.0. According to a most preferable embodiment of the present disclosure, each polyether amine molecule comprises two amine terminal groups attached to the end of the polyether main chain, and does not comprise pendent amine groups. According to a preferable embodiment of the present disclosure, the polyether amine does not comprise substituting groups other than the amine terminal groups.

According to a preferable embodiment of the present disclosure, the reaction between the polyether amine and the glycerin carbonate occurs at an approximately stoichiometric ratio, e.g. the molar ratio between the glycerin carbonate and the amine groups in the polyether amine can be from 0.8 to 1.2, or from 0.9 to 1.1, or from 0.95 to 1.05, or from 0.98 to 1.08, or 1.0. According to a preferable embodiment of the present disclosure, at least 95%, or at least 97%, or at least 98%, or at least 99%, or at least 99.9% or about 100% of the glycerin carbonate and the polyether amine are consumed according to said reaction shown in FIG. 1, hence there is no unreacted glycerin carbonate and polyether amine remained in the resultant polyol compound of the present disclosure. According to a preferable embodiment of the present disclosure, the resultant polyol compound of the present disclosure has a molecular structure shown in FIG. 1 and thus has a hydroxyl functionality of 4. According to a preferable embodiment of the present disclosure, the polyol compound of the present disclosure has an OH number of 50 to 2,000 mg KOH/g, such as from 60 to 1,500 mg KOH/g, or from 80 to 1,200 mg KOH/g, or from 90 to 1,000 mg KOH/g, or from 95 to 700 mg KOH/g, or from 98 to 600 mg KOH/g, or from 100 to 500 mg KOH/g, or from 1 10 to 350 mg KOH/g.

According to a preferable embodiment of the present application, the polyol compound of the present disclosure can be synthesized at a temperature of 20° C. to 80″C, such as 25″C to 70° C., or from 30° C. to 60° C., or from 40 to 50° C., a pressure of 1 to 5 atmospheres, or from 1 to 3 atmospheres, or from 1 to 1.5 atmosphere, for a duration of 10 minutes to 10 hours, or from 0.5 hour to 8 hours, or from 1 hour to 5 hours, or from 1.5 to 4 hours, or from 2 to 3 hours, without the presence of catalyst or any reaction promoters.

According to another preferable embodiment of the present disclosure, the isocyanate-reactive component (B) comprises a blend of the polyol compound formed by the reaction between the carbonate of Formula 1 and a polyamine and, optionally, a second polyol compound. According to a preferable embodiment of the present disclosure, the content of the polyol compound of the present application in the isocyanate-reactive component (B) is from 20 wt% to 100 wt%, or from 25 wt% to 95 wt%, or from 30 wt% to 80 wt%, or can be in the numerical range obtained by combining any two of the following end point values: 15 wt%, 20 wt%, 22 wt%, 25 wt%, 28 wt%, 30 wt%, 35 wt%, 40 wt%, 42 wt%, 45 wt%, 48 wt%, 50 wt%, 52 wt%, 55 wt%, 58 wt%, 60 wt%, 62 wt%, 63 wt%, 65 wt%, 68 wt%, 70 wt%, 72 wt%, 74 wt%, 75 wt%, 77 wt%, 78 wt%, 80 wt%, 82 wt%, 85 wt%, 88 wt%, 90 wt%, 92 wt%, 95 wt%, 98 wt%, 99 wt% and 100 wt%, with the total weight of the isocyanate-reactive component (B) being taken as 100 wt%.

According to various embodiments of the present disclosure, the second polyol compound is not derived from the reaction between a carbonate of Formula 1 and a polyamine, and can be selected from the group consisting of monomeric polyfunctional alcohols, such as C₂-C₁₆ aliphatic polyhydric alcohols comprising at least two hydroxyl groups, C₆-C₁₅ cycloaliphatic or aromatic polyhydric alcohols comprising at least two hydroxyl groups, C₇-C₁₅ araliphatic polyhydric alcohols comprising at least two hydroxyl groups; and polymeric polyols, such as polyester polyols, polyether polyols, polycarbonate polyols, a blend of said polyester polyols and polyether polyols, and a combination thereof. The content of the second polyol is from 0 wt% to 80 wt%, or from 5 wt% to 75 wt%, or from 10 wt% to 70 wt%, or can be in the numerical range obtained by combining any two of the following end point values: 8 wt%, 10 wt%, 12 wt%, 15 wt%, 18 wt%, 20 wt%, 22 wt%, 25 wt%, 28 wt%, 30 wt%, 32 wt%, 35 wt%, 37 wt%, 40 wt%, 42 wt%. 45 wt%, 48 wt%, 50 wt%, 52 wt%. 54 wt%, 55 wt%, 57 wt%, 60 wt%, 62 wt%, 65 wt%, 67 wt%, 70 wt%, 72 wt%, 75 wt% and 80 wt%, with the total weight of the isocyanate-reactive component (B) being taken as 100 wt%.

### The Application of the Adhesive Composition

According to various embodiments of the present disclosure, the two-component adhesive composition of the present disclosure may comprise one or more solvents or can be completely solventless. As disclosed herein, the terms “solvent free”, “solventless” or “nonsolvent”, can be used interchangeably used and shall be interpreted that the mixture of all the raw materials used for preparing the adhesive composition comprise less than 3% by weight, preferably less than 2% by weight, preferably less than 1% by weight, more preferably less than 0.5% by weight, more preferably less than 0.2% by weight, more preferably less than 0.1% by weight, more preferably less than 100 ppm by weight, more preferably less than 50 ppm by weight, more preferably less than 10 ppm by weight, more preferably less than 1 ppm by weight of any organic or inorganic solvents, based on the total weight of the mixture of raw materials. As disclosed herein, the term “solvent” refers to organic and inorganic liquids whose function is solely dissolving one or more solid, liquid or gaseous materials without incurring any chemical reaction. In other words, although some organic compounds, e.g. ethylene glycol and propylene glycol, and water, which are generally considered as “solvent” in the polymerization technology, are used in the preparation of the two-component polyurethane-based adhesive composition, none of them belongs to “solvent” since they mainly function as isocyanate-reactive functional substance, or chain extending agent, etc. by incurring chemical reactions.

According to various embodiments of the present disclosure, the weight ratio between the isocyanate component (A) (i.e. the prepolymer) and the ioscyanate-reactive component (B) is from 100:20 to100: 100. According to a preferable embodiment, said weight ratio can be in the numerical range obtained by combining any two of the following ratios: 100:20, 100:30, 100:40, 100:50, 100:60, 100:70, 100:80, 100:90, and 100:100. One of the technical advantages of the present disclosure is that the bond strength and heat seal strength of the (cured) adhesive prepared by using the adhesive composition of the present disclosure will not be substantially deteriorated by the change in the above stated ratio. For example, the change in the magnitude of the bond strength and heat seal strength [with and without a BIB (boiling in bag) test] of the (cured) adhesive prepared by using the adhesive composition is less than ±20%, or less than ±15%, or less than ±10%, or less than ±8%, or less than ±6%, or less than ±5%, or less than ±3%, or less than ±2%, or less than ±1%, or less than ±0.5%, or less than ±0.3%,when the molar ratio between the prepolymer in the isocyanate component (A) and the polyol compounds in the ioscyanate-reactive component (B) increases or decreases by at most 60%, or at most 55%, or at most 50%, or at most 45%, or at most 40%, or at most 35%, or at most 30%, or at most 25% or at most 20%, or at most 15%, or at most 10%, from the stoichiometric ratio, with the bond strength and heat seal strength of the (cured) adhesive prepared by using the adhesive composition having a stoichiometric ratio being taken as 100%.

As stated above, the isocyanate component (A) and the isocyanate-reactive component (B) are transported and stored separately, combined shortly or immediately before being applied during the manufacture of the laminate article. In some embodiments, both the isocyanate component and the polyol are liquid at ambient temperature. When it is desired to use the adhesive composition, the isocyanate component and the isocyanate-reactive component are brought into contact with each other and mixed thoroughly. Once mixed, polymerization (curing) reaction occurs between the free isocyanate groups in the isocyanate component (A) (preferably the urethane prepolymer) and the hydroxyl groups in the isocyanate-reactive component (B) to form a polyurethane which exhibit the function of adhesive in the adhesive layer between two or more substrates. The adhesive composition formed by bringing the two components into contact can be referred to as a “curable mixture”.

One or more catalysts may be optionally used to promote or accelerate the above stated polymerization reaction for preparing the prepolymer in the isocyanate component (A) and/or the polymerization between the prepolymer of (A) and the isocyanate-reactive component (B). The catalyst may include any substance that can promote the reaction between the isocyanate group and the hydroxyl group. Without being limited to theory, the catalysts can include, for example, glycine salts; tertiary amines; tertiary phosphines, such as trialkylphosphines and dialkylbenzylphosphines; morpholine derivatives; piperazine derivatives; chelates of various metals, such as those which can be obtained from acetylacetone, benzoylacetone, trifluoroacetyl acetone, ethyl acetoacetate and the like with metals such as Be, Mg, Zn, Cd, Pd. Ti, Zr, Sn, As, Bi. Cr, Mo. Mn, Fe, Co and Ni; acidic metal salts of strong acids such as ferric chloride and stannic chloride; salts of organic acids with variety of metals, such as alkali metals, alkaline earth metals, Al, Sn, Pb, Mn, Co, Ni and Cu; organotin compounds, such as tin(II) salts of organic carboxylic acids, e.g., tin(II) diacetate, tin(II) dioctanoate, tin(II) diethylhexanoate, and tin(II) dilaurate, and dialkyltin(IV) salts of organic carboxylic acids, e.g., dibutyltin diacetate, dibutyltin dilaurate, dibutyltin maleate and dioctyltin diacetate; bismuth salts of organic carboxylic acids, e.g., bismuth octanoate; organometallic derivatives of trivalent and pentavalent As, Sb and Bi and metal carbonyls of iron and cobalt; or mixtures thereof. In general, the content of the catalyst used herein is larger than zero and is at most 1.0 wt%, preferably at most 0.5 wt%, more preferably at most 0.05 wt%, based on the total weight of all the reactants.

The adhesive composition of the present disclosure may optionally comprise any additional auxiliary agents and/or additives for specific purposes. In one embodiment of the present disclosure, one or more of the auxiliary agents and/or additives may be selected from the group consisting of other co-catalysts, surfactants, toughening agents, flow modifiers, adhesion promoters, diluents, stabilizers, plasticizers, catalyst de-activators, dispersing agents and mixtures thereof.

A method of producing a laminate article using said adhesive composition is also disclosed. In some embodiments, the adhesive composition, such as the adhesive composition discussed above, is in a liquid state. In some embodiments, the composition is a liquid at 25° C. Even if the composition is solid at 25° C., it is acceptable to heat the composition as necessary to convert it into a liquid state. A layer of the composition is applied to a surface of a substrate or a film. A “substrate/film” is any structure that is 0.5 mm or less in one dimension and is 1 cm or more in both of the other two dimensions. A polymer film is a film that is made of a polymer or mixture of polymers. The composition of a polymer film is, typically, 80 percent by weight or more by weight one or more polymers. In some embodiments, the thickness of the layer of the curable mixture applied to the film is 1 to 5 µm.

In some embodiments, a surface of another substrate/film is brought into contact with the layer of the curable mixture to form an uncured laminate. The adhesive composition may be applied by conventional lamination machine, e.g. Labo-Combi 400 machine from Nordmeccanica. The curable mixture is then cured or allowed to cure. The uncured laminate may be subjected to pressure, for example by passing through nip rollers, which may or may not be heated. The uncured laminate may be heated to speed the cure reaction. Suitable substrates/films include paper, woven and nonwoven fabric, metal foil, polymers, and metal-coated polymers. Films optionally have a surface on which an image is printed with ink: and the ink may be in contact with the adhesive composition. In some embodiments, the substrates/films are polymer films or metal-coated polymer films, and more preferred are polymer films.

The laminate article disclosed herein can be cut or otherwise shaped so as to have a shape suitable for any desired purpose, such as packaging material.

Although the above general description and the following examples mainly focus on a two-component PU-based adhesive composition, the unique hydroxyl compound of the present disclosure can be used as an isocyanate-reactive compound for any other polyurethane-based products, such as coating, paint, insulation material, packaging material, foam material, etc., and impart the above stated technical advantages to these products.

### EXAMPLES

Some embodiments of the invention will now be described in the following Examples, wherein all parts and percentages are by weight unless otherwise specified. However, the scope of the present disclosure is not, of course, limited to the formulations set forth in these examples. Rather, the Examples are merely inventive of the disclosure.

The information of the raw materials used in the examples is listed in the following table 1:

TABLE 1

Raw materials used in the examples

| | | |
|---|---|---|
| Grade name | Characterization | Vendor |
| Jeffamine D400 | Polyether amine with a Mn of 430 and an amine functionality of 2 | Huntsman |
| Jeffamine D2000 | Polyether amine with a Mn of 2,000 and an amine functionality of 2 | Huntsman |
| Glycerine carbonate | 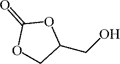 | Huntsman |
| VORANOL™ CP450 | Polyether polyol having a Mn of 450 and a hydroxyl number of 378 | The Dow Chemical Company |
| Bester™ 1093 | Polyester polyol | The Dow Chemical Company |
| MorFree™ 698A | A NCO terminated prepolymer with NCO%=14.5 | The Dow Chemical Company |

### Synthesis Example 1: The Synthesis of the Polyol Compound of the Present Disclosure With Different Amines

### A. The Preparation of Polyol Compound With a Polyether Diamine Having Lower Molecular Weight

25.4 gram (0.2 mole) of glycerol carbonate was added into a flask equipped with stirring blade and water bath, and was heated to a temperature of 50° C. 43 gram (0.1 mole) of JEFFAMINE D400 was heated to about 50° C. and slowly added into the flask. The flask was kept at a constant temperature of about 50° C. under stirring for 2 hours. The resultant product was referred as HF1.

The product HF1 was characterized with GPC (Gel Permeation Chromatography). In particular, certain amount of HF1 was dissolved in THF to produce a clear solution having a concentration of 10 mg/mL. The solution was filtered through a 0.45 µm PTFE membrane, then 50 µL of the solution was injected into an Agilent 1200 GPC apparatus. The GPC apparatus comprises two mixed E columns (7.8 × 300 mm) having a column temperature of 35° C. and an Agilent refractive index detector operated at a temperature of 35° C. The mobile phase is tetrahydrofuran and flows through the column at a flow rate of 1.0 mL/min. The detection data was collected and treated with an Agilent GPC software was normalized with a calibration curve obtained by using a PL Polystyrene Narrow standards (Part No.:2010-0101) with a polyol equivalent molecular weight ranging from 11450 to 129 g/mol. The resultant molecular weight distribution plot of HF1 was shown in FIG. 2. According to the GPC characterization, HF1 has a polyol equivalent number molecular weight (Mn) of 658 and had a hydroxyl functionality of 4.0.

The product HF1 also exhibits a hydroxyl number (measured according to ASTM D6342:2008) of about 330 mg KOH/g and a viscosity (measured according to GB-T 12008.8-1992) of 1600cps at 70° C.

### B. The Preparation of Polyol Compound of the Present Disclosure With a Polyether Diamine Having Higher Molecular Weight

25.4 gram (0.2 mole) of glycerol carbonate was added into a flask equipped with stirring blade and water bath, and was heated to a temperature of 50° C. 200 gram (0.1 mole) of JEFFAMINE D2000 was heated to about 50° C. and slowly added into the flask. The flask was kept at a constant temperature of about 50° C. under stirring for 2 hours. The resultant product was referred as HF2.

The product HF2 was similarly characterized with GPC and exhibits a polyol equivalent number average molecular weight (Mn) of 1403 and had a hydroxyl functionality of 4.0. Besides, the product HF2 also exhibits a hydroxyl number of about 110 mg KOH/g and a viscosity of 4400 cps at 25° C.

### C. The Preparation of Polyol Compound With a Monomeric Diamine

A polyol compound was prepared by basically following the procedures for preparing the HF2, except that the JEFFAMINE D2000 was replaced with 1 mole of ethylene diamine. The resultant product was referred as HF3 and had a hydroxyl functionality of 4.0.

The product HF3 also exhibits a hydroxyl number of about 296 mg KOH/g and a viscosity (measured according to GB-T 12008.8-1992) of 260 cps at 25° C.

### Examples 1-6 and Comparative Examples 1-2

HF1, HF2 and HF3 were mixed with a polyether polyol to form the polyol component (B) as shown in the following Table 2, and these polyol components (B) were used in the inventive examples 1 to 6. A comparative polyol component (B) was also prepared by mixing a polyether polyol with a polyester polyol and used in the two comparative examples.

TABLE 2

The formulations of the polyol component (B)

| | Composition and loading ratio | OH number (mgKOH/g) |
|---|---|---|
| B1 | CP450/HF1=35/65 | 363 |
| B2 | CP450/HF2=35/65 | 200 |
| B3 | CP450/HF3=35/65 | 625 |
| B4 | CP450/Bester 1093=35/65 | 240 |

The adhesive compositions of Examples 1 to 6 and Comparative Examples 1 to 2 were synthesized according to the formulations listed in Table 3, and the bond strength (BS) and heat seal strength (HS) thereof were characterized by using the following technologies.

TABLE 3

The formulation and characterization of Examples 1-6 and Comparative Examples 1-2

| | | PET/PE60 | | | |
|---|---|---|---|---|---|
| | | BS | HS | BS after BiB | HS after BiB |
| Example 1 | MorFree™698A/B1=100:33 | 3.2 | 47 | 2.5 | 37 |
| Example 2 | MorFree™698A/B1=100:50 | 3.5 | 48 | 2.9 | 46 |
| Example 3 | MorFree™698A/B2=100:55 | 2.7 | 49 | 2.9 | 45 |
| Example 4 | MorFree™698A/B2=100:82 | 2.8 | 20 | 2.9 | 21 |
| Example 5 | MorFree™698A/B3=100:21 | 2.8 | 53 | 2.9 | 52 |
| Example 6 | MorFree™698A/B3=100:29 | 2.7 | 50 | 2.6 | 50 |
| Comparative Example 1 | MorFree™698A/B4=100:50 | 2.8 | 52 | 2.3 | 52 |
| Comparative Example 2 | MorFree™698A/B4=100:75 | 2.6 | 50 | tunnel | tunnel |

The polyol components prepared in Table 2 were paired with Dow commercial product (NCO prepolymer) MorFree™698A at the ratios shown in Table 3 to form the adhesives and subject to performance evaluation.

Laminates were prepared with these adhesives in a Labo-Combi 400 machine from Nordmeccanica under the following processing conditions: line speed was set as 120 mpm temperature of transfer roller was 45° C., nip temperature was set as 60° C., and coating weight was set as 1.8 gsm. Different substrates were selected to form PET/PE60 as testing laminate structures, which were characterized with the following technologies.

### Test Methods

### Bond Strength (BS)

Laminates prepared with the adhesive compositions, were cut into 15 mm width strips for T-peel test under 250 mm/min crosshead speed using a 5940 Series Single Column Table Top System available from Instron Corporation. During the test, the tail of each strip was pulled slightly by fingers to make sure the tail remained 90 degree to the peeling direction. Three strips for each sample were tested and the average value was calculated. Results were represented with the unit of N/15mm. A higher value represents a better the bond strength.

### Heat Seal Strength (HS)

Laminates prepared with the adhesive compositions were heat-sealed in a HSG-C Heat-Sealing Machine available from Brugger Company under 140° C. seal temperature and 300N pressure for 1 second, then cooled down and cut into 15 mm width strips for heat seal strength test under 250 mm/min crosshead speed using a 5940 Series Single Column Table Top System available from Instron Corporation. Three strips for each sample were tested and the average value was calculated. Results were represented with the unit of N/15 mm. A higher value represents a better heat seal strength.

### Boil in Bag (BiB)

Laminates prepared with the adhesive compositions were cut into 8 cm×12cm pieces which were heat sealed to form a bag with water enclosed therein. Then the bag was immersed in boiling water and held for 30 minutes, during which the bag was kept completely immersed in the boiling water. After the 30 minute boiling, the bag was inspected for any defects such as tunneling, de-lamination, or leakage, and the extents of said defects, if any, were recorded. A sample that passed the test should show no evidence of tunneling, de-lamination, or leakage. The bag was opened, emptied and cooled down, and then cut into 15 mm width strips to test the T-peel bonding strength and heat seal strength thereof in an Instron 5943 machine. Three strips for each sample were tested and the average value was calculated.

It can be seen from Table 3 that all the inventive examples comprising HF1, HF2 and HF3 exhibit superior HS and BS which will not be deteriorated to an unacceptable extent no matter how the ratio between the two components changes, while both of the comparative examples exhibit much higher deterioration in both HS and BS when the ratio between component (A) and component (B) exceeds 100:50 and will form tunnel during the boil in bag (BiB) treatment.

What is claimed is:

1. A polyol compound formed by a reaction between a carbonate compound represented by Formula I and a polyamine compound comprising at least two amine groups, 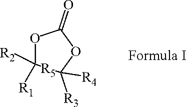
wherein each of R₁, R₂, R₃ and R₄ are identical with each other or different from each other, and are independently selected from the group consisting of hydrogen, linear or branched C₁ to C₆ alkyl, linear or branched C₁-C₆ alkoxy, hydroxyl, halogen and hydroxyl substituted-C₁-C₆ alky, or R₁ is directly linked with R₃ so that a combination of R₁, R₃, R₅ and the carbon atoms attached thereto form a C₅-C₈ cycloalkyl group substituted with at least one hydroxyl,
R₅ is a direct covalent bond, a methylene group, a 1,2-ethylene group, or a 1,3-propylene group, wherein the methylene group, 1,2-ethylene group and 1,3-propylene group are optionally substituted with at least one substituting group selected from the group consisting of C₁-C₆ alkyl, C₁-C₅ alkoxy, hydroxyl, halogen and hydroxyl-C₁-C₆ alky, and
wherein the polyol compound comprises at least two hydroxyl groups.

2. The polyol compound according to claim 1, wherein the carbonate compound is selected from the group consisting of glycerin carbonate, 1-methyl-glycerin carbonate, 2-methyl-glycerin carbonate, 3-methyl-glycerin carbonate, 1,1-dimethyl-glycerin carbonate, 2,2-dimethyl-glycerin carbonate, 1,2-dimethyl-glycerin carbonate, 1-ethyl-glycerin carbonate, 1,1-diethyl-glycerin carbonate, 2-ethyl-glycerin carbonate, 2,2-diethyl-glycerin carbonate, 1-propyl-glycerin carbonate, 1-butyl-glycerin carbonate, 1,2,3,4-tetrahydroxybutane carbonate, 1,2,3,4,5-pentahydroxypentane carbonate, 1,2,3,4,5,6-hexahydroxyhexane carbonate, trimethyolmethane carbonate, trimethyolethane carbonate, trimethylolpropane carbonate, pentaerythritol carbonate, and combinations thereof.

3. The polyol compound according to claim 1, wherein the polyamine compound is selected from the group consisting of piperazine, C₂-C₁₆ aliphatic diamine or triamine, C₄-C₁₅ cycloaliphatic or aromatic diamine or triamine, C₇-C₁₅ araliphatic diamine or triamine, diaminated or triaminated C₂-C₈ aliphatic alcohol, polyether diamine, polyester diamine, and combinations thereof.

4. The polyol compound according to claim 1, wherein the reaction between the carbonate compound and the polyamine compound occurs with a molar ratio between the carbonate compound and polyamine compound of 5:1 to 1:5.

5. A method for preparing the polyol compound according to any one of claims 1, comprising a step of reacting a carbonate compound represented by Formula I with a polyamine compound comprising at least two amine groups to form the polyol compound having at least two hydroxyl groups, 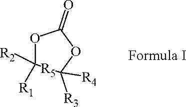
wherein each of R₁, R₂, R₃ and R₄ are identical with each other or different from each other, and are independently selected from the group consisting of hydrogen, linear or branched C₁ to C₆ alkyl, linear or branched C₁-C₅ alkoxy, hydroxyl, halogen and hydroxyl-C₁-C₆ alky, or R₁ is directly linked with R₃ so that a combination of R₁, R₃, R₅ and the carbon atoms attached thereto forms a C₅-C₈ cycloalkyl group substituted with at least one hydroxyl group, and
R₅ is a direct covalent bond, a methylene group, a 1,2-ethylene group, or a 1,3-propylene group, wherein the methylene group, 1,2-ethylene group and 1,3-propylene group are optionally substituted with C₁-C₆ alkyl, C₁-C₅ alkoxy, hydroxyl, halogen and hydroxyl-C₁-C₆ alky.

6. An adhesive composition, comprising:
(A) an isocyanate component comprising a prepolymer having two or more free isocyanate groups; and
(B) an isocyanate-reactive component comprising the polyol compound according to claim 1.

7. The adhesive composition according to claim 6, wherein the weight ratio between the prepolymer and the polyol compound is from 100:20 to 100:100.

8. The adhesive composition according to claim 6, wherein the isocyanate-reactive component further comprises at least one second polyol compound selected from the group consisting of polycarbonate polyol, polyether polyol, polyester polyol, and combinations thereof.

9. The adhesive composition according to claim 6, wherein the prepolymer is derived from the reaction of an isocyanate compound having at least two isocyanate groups with at least one second isocyanate-reactive compound,
wherein the isocyanate compound is selected from the group consisting of C₄-C₁₂ aliphatic isocyanate comprising at least two isocyanate groups, C₆-C₁₅ cycloaliphatic or aromatic isocyanate comprising at least two isocyanate groups, C₇-C₁₅ araliphatic isocyanate comprising at least two isocyanate groups, carbodiimide modified isocyanates, and a combination thereof, and
the second isocyanate-reactive compound is selected from the group consisting of C₂-C₁₆ aliphatic polyhydric alcohol comprising at least two hydroxyl groups, C₆-C₁₅ cycloaliphatic or aromatic polyhydric alcohol comprising at least two hydroxyl groups, C₇-C₁₅ araliphatic polyhydric alcohol comprising at least two hydroxyl groups, polyester polyol, polyether polyol, polycarbonate polyol and combinations thereof.

10. The adhesive composition according to claim 6, wherein the prepolymer is derived from the reaction of an isocyanate compound having at least two isocyanate groups with a polyol compound formed by reaction between a carbonate compound represented by Formula 2 and a polyamine compound comprising at least two amine groups, 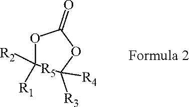
wherein each of R_{1,} R_{2,} R₃ and R₄ are identical wish each other from each other, and are independently selected from the group consisting of hydrogen, linear or branched C₁ to C₆ alkyl, linear or branched C₁-C₆ alkoxy, hydroxyl, halogen and hydroxyl subsituted-C₁-C₆ alky, or R₁ is directly linked with R₃ so that a combination of R₁, R₃, R₅ and the carbon atoms attached thereto form a C₅-C₈ cycloalkyl group substituted with at least one hydroxyl
R₅ is a direct covalent bond, a methylene group, a 1,2-ethylene group, or a 1,3-propylene group, wherein the methylene group, 1,2-ethylene group and 1,3-propylene group are optionally substituted with at least one substituting group selected from the group consisting of C₁₋C₆ alkyl C₁-C₅ alkoxy, hydroxyl, halogen and hydroxyl-C₁-C₆ alky, and
wherein the polyol compound comprises at least two hydroxyl groups, and
wherein the isocyanate compound is selected from the group consisting of C₄-C₁₂ aliphatic isocyanate comprising at least two isocyanate groups, C₆-C₁₅ cycloaliphatic or aromatic isocyanate comprising at least two isocyanate groups, C₇-C₁₅ araliphatic isocyanate comprising at least two isocyanate groups, carbodiimide modified isocyanates, and a combination thereof.

11. The adhesive composition according to claim 6, wherein the adhesive composition comprises solvent or is solventless.

12. A method for preparing a laminate article with the adhesive composition according to claim 6, comprising the steps of
providing a first substrate and a second substrate,
mixing the isocyanate component with the isocyanate-reactive component to form a curable mixture;
adhering the first substrate to the second substrate by using a layer of the curable mixture; and
curing the curable mixture, or allowing it to cure.

13. A laminate article comprising at least two substrates and an adhesive layer sandwiched therebetween, wherein the adhesive layer is formed by the reaction between the isocyanate component and the isocyanate-reactive component of the adhesive composition according to claim 6.
